(19) United States
(12) Patent Application Publication
(10) Pub. No.: US 2016/0266134 A1
(43) Pub. Date: Sep. 15, 2016

(54) CROSSLINKED POLYMER STATIONARY PHASE FOR CHROMATOGRAPHY

(71) Applicant: CLEMSON UNIVERSITY, Clemson, SC (US)

(72) Inventors: R. Kenneth Marcus, Clemson, SC (US); Liuwei Jiang, Central, SC (US)

(21) Appl. No.: 15/067,339

(22) Filed: Mar. 11, 2016

Related U.S. Application Data
(60) Provisional application No. 62/131,431, filed on Mar. 11, 2015.

(51) Int. Cl. G01N 33/68; G01N 33/72; C12Q 1/34

(52) U.S. Cl. CPC G01N 33/6803; C12Q 1/34; G01N 33/721; G01N 2333/765; G01N 2333/936; G01N 2333/805

(57) ABSTRACT

Separation technologies and a support/separation phases for use therein. A surface of a support phase can be modified to include a crosslinked polymer network as stationary phase to perform separation of one or more species from a liquid in highly efficient separations based on chemical interactions, i.e., chromatography. Optionally, the support phase can employ polymer fibers having channels extending axially along their surfaces. The use of the support phase to support the crosslinked stationary phase can be used in one embodiment in the process of performing micro-scale separations.

### CROSS REFERENCE TO RELATED APPLICATION

This application claims filing benefit of U.S. Provisional Patent Application Ser. No. 62/131,431 having a filing date of Mar. 11, 2015 entitled “Crosslinked Polymer Stationary Phase for Chromatography Columns,” which is incorporated herein by reference.

### STATEMENT WITH REGARD TO FEDERALLY SPONSORED RESEARCH AND DEVELOPMENT

This invention was made with government support under grant #1307078 awarded by the National Science Foundation. The government has certain rights in the invention.

### BACKGROUND

At present, liquid-phase chemical separations are usually performed in “columns” prepared by the packing of metal tubes with spherical beads that are composed of either silica or polystyrene and have diameters of ˜2 to 50 μm. The more or less inert beads provide solid supports that are chemically modified to produce a surface having targeted chemical characteristics. For example, in performing reversed-phase liquid chromatography, long carbon chains (C-18) are affixed to the surfaces of the beads to produce a hydrophobic surface for the separation of non-polar organics. As an alternative mechanism to affect separations, electrostatic interactions between solute molecules in the mobile phase can occur with charged ligand moieties on the support phase surface, a process known as ion exchange chromatography (IEC or IEX).

Effective separations require dense packing of the beads into these columns to avoid dead-volume, which is any location within the column where turbulence can occur and interactions between molecules in the liquid and the surfaces of the beads are absent. As a consequence of dense packing, high driving pressures (2,000 to 40,000 psi) are required to overcome the backing pressures that otherwise would prevent the liquid phase from moving through the densely packed columns.

Alternatively, highly porous “monoliths” are formed within the columns to generate high surface areas for interaction with the species that flow through the columns. Here, column-to-column irreproducibility, limitation of column sizes and a limited set of stationary phase chemistries can be restrictive. In the case of so-called “prep-scale” separations, the capital costs associated with producing large volume columns and the demands on the system hydraulics (i.e. pumps) are very high.

Capillary-channeled polymer fibers have been extensively studied for separations such as bio-macromolecule separations. Such fibers are generally manufactured by melt-extruding a polymeric composition with the desired channeled morphology including multiple capillary channels extending to the whole length of the fiber. The morphology of the channeled fibers gives the fibers a much greater surface area as compared to circular cross-sectional fibers with the same diameters. When packed in columns, capillary-channeled fibers can self-align and yield a monolith-like structure with open, parallel channels. These channels provide excellent fluid transportation property and low mass transfer resistance in the columns. It is believed that the high column permeability, low mass transfer resistance and the low cost of the channeled fiber chromatography columns make them promising choices for fast separations at various scales.

The base polymer identity of the fibers provides different surface chemistries, and thus, protein separations are affected in different modes. For instance, polyethylene terephthalate (PET) and polypropylene (PP) channeled fibers have been used for reversed phase (RP) chromatography, due to their hydrophobic surfaces. Nylon 6 channeled fibers, with amine and carboxylic acids present on their surfaces, have been used in mixed-mode ion-exchange and hydrophobic interaction chromatography (HIC) separations. Unfortunately, the base polymers useful in forming the fibers are not always ideal for the stationary phase of a separation column, as interaction is dictated by the surface chemistry of the native fibers.

While the above describes improvement in the art, room for further improvement exists.

### SUMMARY

According to one embodiment, disclosed is an apparatus that includes a fluid conduit having a first end and a second end that is disposed opposite the first end. The apparatus also includes a polymeric support phase that is disposed within the conduit between the first end and the second end. The support phase includes a crosslinked polymer as a stationary phase, the crosslinked polymer being at the surface of the polymeric support phase.

In one particular embodiment, the polymeric support phase can include capillary-channeled polymeric fibers. Accordingly, in one embodiment, disclosed is a polymeric capillary-channeled fiber that includes a crosslinked polymer as a stationary phase at a surface of the channeled fiber.

According to one embodiment, disclosed is a method for separating a species from a fluid. The method can include moving a fluid through the conduit that contains a support phase and a stationary phase, the support phase including a polymeric structure (e.g., a capillary-channeled fiber) and the stationary phase including a crosslinked polymer at a surface of the polymeric structure. Upon moving the fluid through the conduit, the species of interest can preferentially adhere to the stationary phase.

Additional objects and advantages of the invention will be set forth in part in the description which follows, and in part will be obvious from the description, or may be learned by practice of the invention. The objects and advantages of the invention may be realized and attained by means of the instrumentalities and combinations particularly pointed out in the appended claims.

### BRIEF DESCRIPTION OF THE DRAWINGS

The disclosed subject matter may be better understood with reference to the drawings in which:

FIG. 1 is a cross-sectional representation of a liquid analyte flowing through channeled fibers placed in a single column formed by a tube and including an expanded view window showing the end-on shape of representative fibers and the potential irregular packing of the fibers in the column.

FIG. 2A is a photograph of an enlarged side plan view of an intermediate portion of an embodiment of a channeled polyester fiber that can be used in a column of a chromatograph.

FIG. 2B is a photograph of enlarged side plan views of end portions of presently preferred embodiments of two channeled polyester fibers that can be used in a column of a chromatograph.

FIG. 3 is a photograph of an enlarged side plan view of an intermediate portion of an embodiment of a channeled polypropylene fiber that can be used in a column of a chromatograph.

FIG. 4 schematically illustrates the chemical functionality of a surface of nylon 6.

FIG. 5 schematically illustrates the surface modification of a poly(ethylene terephthalate) (PET) surface with poly(ethylene imine) (PEI).

FIG. 6 presents scanning electron microscope (SEM) images of native and modified PET channeled fibers.

FIG. 7A is an AFM image of a native PET channeled fiber surface.

FIG. 7B is an AFM image of a PET fiber modified with an uncrosslinked PEI at the surface.

FIG. 7C is an AFM image of a PET fiber modified with a crosslinked PEI at the surface.

FIG. 8 is a graph illustrating column back-pressures as a function of mobile phase flow rate for a variety of different columns.

FIG. 9A presents the protein loading and the elution transient under reversed phase and anion-exchange elution conditions for two different columns using a first elution buffer (RP elution).

FIG. 9B presents the protein loading and the elution transient under reversed phase and anion-exchange (AEC) elution conditions for two different columns using a second elution buffer (AEC elution).

FIG. 10 is a graphical presentation of the effect of protein concentration on the dynamic loading for different channeled fiber columns at a flow rate of 1 mL/min. The lines reflect the fitting to a Langmuir isotherm model to generate dynamic binding capacities.

FIG. 11 illustrates the effect of the load linear velocity on the dynamic loading capacity of a protein on different channeled fiber columns. The protein loading concentration was 1 mg/mL.

FIG. 12 presents chromatograms for the AEC separation of three different proteins on a column as described herein at four different flow rates.

FIG. 13A is a chromatogram of a three-protein separation on a channeled fiber column at different mobile phase flow rates while maintaining a constant volumetric gradient phase.

FIG. 13B is a chromatogram of a three-protein separation on another channeled fiber column at different mobile phase flow rates while maintaining a constant volumetric gradient phase.

FIG. 13C is a chromatogram of a three-protein separation on another channeled fiber column at different mobile phase flow rates while maintaining a constant volumetric gradient phase.

FIG. 14 compares the p/ values of the surface functionality of nylon 6 fibers and three test proteins. The pKₐ of the amine and acid functionalities were calculated by Advanced Chemistry Development Software V11.02 using N-(6′-aminocaproyl)-6-aminocaproic acid as the model molecule

FIG. 15A presents a chromatogram of a three-protein separation on a channeled fiber column at a constant mobile phase flow rate and different gradient rates.

FIG. 15B presents a chromatogram of a three-protein separation on another channeled fiber column at a constant mobile phase flow rate and different gradient rates.

FIG. 15C presents a chromatogram of a three-protein separation on another channeled fiber column at a constant mobile phase flow rate and different gradient rates.

### DETAILED DESCRIPTION

Reference now will be made in detail to embodiments of the disclosed subject matter, one or more examples of which are illustrated in the accompanying drawings. Each example is provided by way of explanation of the subject matter, not limitation of the subject matter. In fact, it will be apparent to those skilled in the art that various modifications and variations can be made in the disclosed embodiments without departing from the scope or spirit of the subject matter. For instance, features illustrated or described as part of one embodiment, can be used on another embodiment to yield a still further embodiment. Thus, it is intended that the present disclosure cover such modifications and variations as come within the scope of the appended claims and their equivalents.

In general, disclosed herein are separation technologies utilizing a support phase in which a surface of the support is chemically modified to effect highly efficient IEC/IEX separations based on chemical interactions, i.e., chromatography. In one particular embodiment, the support phase can employ polymer fibers having channels extending axially along their surfaces in prep-scale separations of mixtures carried in liquid media. More specifically, the support phase can be modified to include a crosslinked stationary phase to perform separation of one or more species from a fluid. For instance, polymer fibers having channels extending axially along their surfaces can be used as support phases to support a crosslinked stationary phase in the process of performing micro-scale separations.

The separation materials and methods can be utilized to selectively extract targeted specie(s) from a fluid. For instance, the materials and methods can be utilized to selectively extract organic molecules (e.g., ionic molecules) or cellular matter such as bacteria from growth media. In one embodiment, the separation materials and methods can be utilized to immobilize cell matter and/or bacteria.

In one embodiment, the chemical modification strategy can be utilized to modify any form of chromatographic support phase to achieve a surface (i.e., surface stationary phases) of the support phase to affect solute separations via ion exchange processes. In this regard, a structure formed of a polymeric composition can serve as a support phase, and the chemical functionality can be utilized to affect further chemical functionalization to create a chemically-tuned stationary phase. In this regard, the support provides a physical structure conducive to fluid and solute movement, taking advantage of the added chemical functionality of the stationary phase to elicit the varying degrees of solute/surface interactions that allow chromatographic separation of solution solutes.

In one embodiment the support phase can include polymer fibers having a non-circular cross-sectional geometry. These fibers can be used as stationary phase support materials for liquid chromatography separations. The cross-sectional geometry arises from channels extending axially and continuously along the fiber surface over the entire length of the fiber. Each fiber desirably can have a uniform nominal diameter (measured at the largest cross-sectional point-to-point location) in the range of about 20 to about 50 micrometers. Use of the surface-channeled fibers can allow for a wide range of liquid flow rates with very low backing pressures.

In one embodiment, a single surface channeled fiber can be used in single fiber separations. For example a column structure can take the form of a single fiber in-laid in a micro-machined device. In another embodiment, bundles of fibers having a channeled cross-sectional geometry and carrying the stationary phase materials can be packed into columns.

Variation in the polymeric composition used in forming the support phase can permit the “chemical tuning” of the separation process. Through combination with a crosslinked separation phase at the surface, a surface-channeled fiber can provide very efficient solution mass transfer with very reactive surface areas.

Contemplated specific applications include use of the separation phase materials in: analytical separations such as liquid chromatography (HPLC, cap-LC); prep-scale separations of specialty chemicals; micro-scale separations; single fiber separations; extraction of selected organic molecules/ions from solution; purification of liquid streams (process waste, drinking water, pure solvents); selective extraction of cell matter and bacteria from growth media; and immobilization of cell matter and bacteria. Potential applications for the invention can include analytical instrumentation; specialty chemicals; and pharmaceutical companies. Demand for the product can be based on its advantages in attaining high throughput and productivity.

While much of the below discussion is directed to separation materials utilizing as a support phase one or more surface channeled polymer fibers, it should be understood that the support phase is not limited to the surface-channeled fibers, and other geometries are contemplated herein, including, without limitation, circular fibers, hollow fibers, solid and/or porous beads of any desired geometry, monolithic support phases, including porous and channeled monoliths, and so forth.

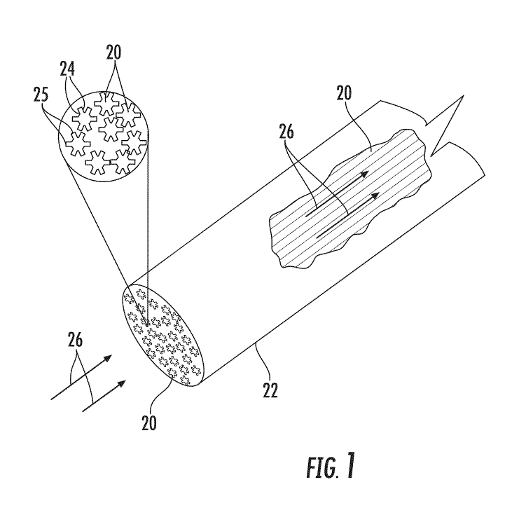

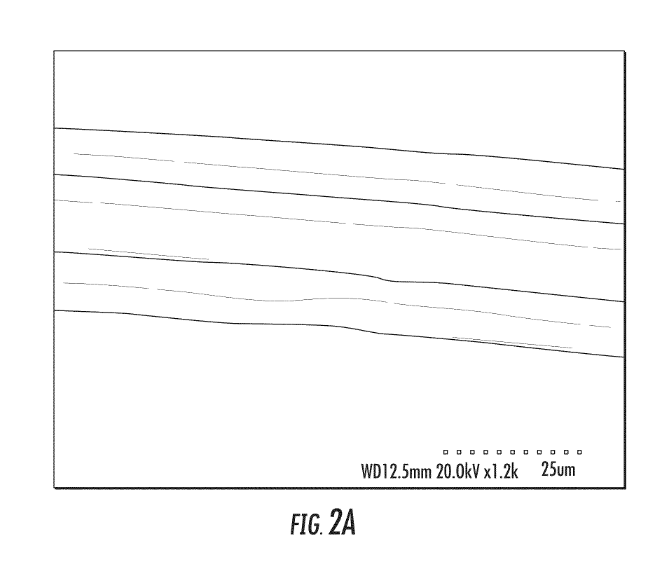

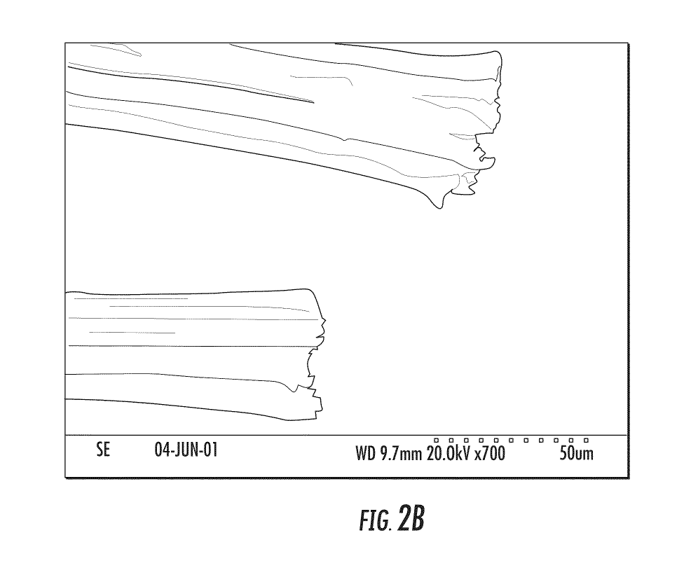

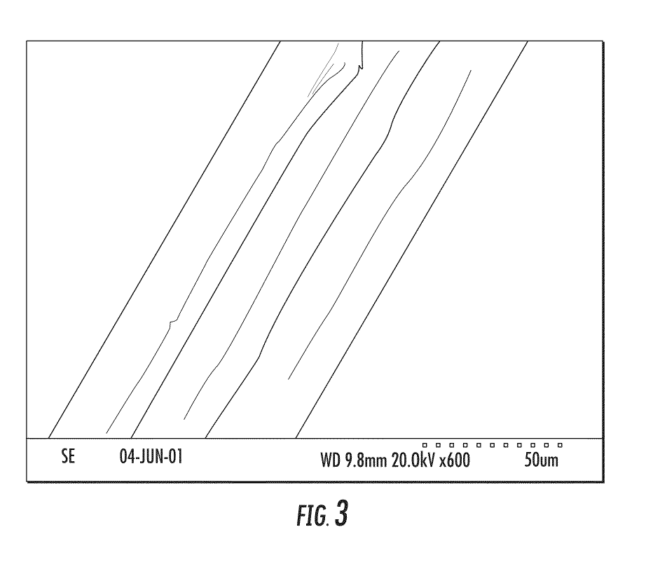

As shown in FIG. 1-FIG. 3, in one embodiment, the support phase can encompass surface-channeled fibers. As illustrated, bundles of surface-channeled polymer fibers 20 can be packed into a column 22 that is formed in one embodiment by a stainless steel tube having a uniform circular inside diameter of 0.25 inches and a length of 12 inches. The dimensions of the column 22 can be any size that is used in the practice of chromatography. Desirably, the length of each fiber 20 is substantially the same as the length of the column 22 and is disposed to extend within the column 22 over substantially the entire length of the column 22. However, fibers 20 that have lengths that are shorter than the length of the column 22 may be used, but are not preferred.

As shown schematically in cross-section in the expanded view window of FIG. 1, each fiber strand 20 has six co-linear channels 24 extending the entire length of the exterior surface of the fiber 20. Each channel 24 is defined by a pair of opposed walls 25 that extend generally and longitudinally and form part of the exterior surface of the fiber 20. Desirably, these channels 24 and walls 25 extend down the entire length of the fiber 20 parallel to the longitudinal axis of the fiber 20 and are co-linear on each fiber 20. This produces de facto substantially the same co-linear channels 24 along the entire length of the column 22. It should be understood that the particular shapes of the capillary-channeled fibers illustrated in FIG. 1 are not a requirement of the present disclosure. In particular, the number and/or cross-sectional shape of the capillaries as well as the overall shape of the capillary-channeled fibers can vary from that shown in the figures.

In an alternative embodiment, the channels 24 can be configured to wrap around the length of the fiber 20 in a helical fashion. However, substantially all of the channels 24 can be co-linear on each fiber 20, and in one embodiment all of the channels 24 of the fibers 20 within each column 22 can follow a helix pattern that has the same pitch. The pitch is the number of complete turns of the channel 24 around the circumference of the fiber 20 per unit of length of the fiber 20. This also produces de facto substantially the same co-linear channels 24 along the entire length of the column 22.

Additionally, in the course of packing the fibers 20 into a bundle that lays along the entire length of the column 22, whether the individual fibers have purely linear channels 24 or helical ones, it is possible that one or more, even all, of the fibers 20 in the bundle will rotate about its/their own axis or the axis of the column 22 over the entire length of the column. In other words, the surface-channeled fibers 20 may twist as they lay from one end of the column 22 to the opposite end. Accordingly, the channels 24 and walls 25 also may twist somewhat.

In some embodiments, a device can be provided to move fluid through the column, e.g., the column 22 of FIG. 1, and thus through the channels 24 of the fibers 20. A pump (not shown) is typically provided for this purpose. The flow of liquid through the column 22 is schematically indicated by the arrows designated by the numeral 26 in FIG. 1. A portion of the column 22 is cut away in the view shown in FIG. 1 for the purpose of illustrating the flow of liquid 26 through the column 22 along the fibers 20 arranged with their longitudinal axes parallel to the longitudinal axis of the column 22.

In some applications, movement of a fluid may be effected without a device that is separate from the support phase. In such embodiments, the fluid can move through the channels 24 of the fibers 20 solely by capillary action of the channels 24 of the fibers 20.

Advantageous in the use of channeled polymer fibers 20 as support phase materials is their very high surface area-to-volume ratios versus circular cross section fibers. The number of channels 24 may vary from the six that are shown schematically in FIG. 1. The shape and the number of channels 24 can be dependent on achieving the desired attribute of very high surface area-to-volume ratios. In this regard, the helical channels 24 in which the fibers can define a twist along the length of the column can pack more surface area into that column than the linear channels.

Another advantage of using channeled polymer fibers 20 for separations is the fact that they generate very low backing pressures (e.g., about 500 to about 800 psi for linear channels) for normal chromatography flow rates (e.g., about 0.5 mL/min to about 3 mL/min). The lower backing pressure produced in a column 22 containing channeled polymer fibers 20 relative to the backing pressure produced in the conventional column containing beads, is believed to be due to the parallel-running channels 24. The ability to use fibers 20 of any desired length, while encountering relatively low backing pressures, would suggest great potential for using columns 22 of these channeled polymer fibers 20 in prep-scale separations or for waste remediation in a variety of industries.

There are different fabrication approaches to form channeled polymer fibers 20 of the sort demonstrated herein. The process can generally include any that is amenable to a polymer composition that can be spin-melted. For example, channeled fibers 20 may be melt spun from a polymeric composition that includes any of a number of different polymer precursors, including polypropylene precursors, polyester precursors, polyaniline precursors, precursors composed of polylactic acid, and the like, as well as combinations of different polymers. In one embodiment, the polymeric composition can include the formed polymers (as opposed to polymeric precursors that can be polymerized during/following formation of the fibers. A polymeric composition can include, without limitation, polymers such as poly(ethylene terephthalate) (PET), polyamide (e.g., nylon 6) or polypropylene (PP). In general use, these channeled polymer fibers 20 can have a very strong wicking action for a variety of liquids, including water.

Liquid chromatography is based on the relative partitioning (driven by a variety of chemical interaction modalities) between a separation phase and the solution phase, and so relative retention characteristics can be an excellent indicator of the actual interactions. The use of different combinations of support/stationary phases, analyte/solutes, and mobile phases provides empirical insights into the retention processes.

For instance, nylon fibers have three different functional groups on the surface as depicted in FIG. 4: amide, primary amine, and carboxylic acid. The amine and carboxylic acid polymer end groups provide the electrostatic interaction sites for ion exchange, while the amide moiety enhances the hydrophilicity of the nylon surface, reducing the hydrophobic interactions of proteins. The presence of both anion (amine) and cation (carboxylic acid) (i.e., zwitterionic) exchangers at neutral pH yields a mixed-mode separation scenario, dependent upon the isoelectric point of the respective proteins (though this is an admitted oversimplification).

According to the present disclosure, the polymeric support phase can be modified to include a crosslinked stationary phase at a surface. Studies can evaluate the support phase modified to include the crosslinked stationary phase in analytical and prep-scale separations. The actual experimental procedure can involve a two-prong approach including: 1) development of column packing methodology, and 2) investigations into on-column derivatization of the base polymer support phase using the described chemistries which form the corpus of the disclosure.

In one embodiment, manual packing can be utilized to locate the polymer fibers into the column, e.g., a steel tube column 22 as depicted schematically in FIG. 1. However, a reliably reproducible way of packing the columns with the support/separation phases can be utilized in order to mass-produce columns.

In one embodiment, radial compression technologies can be employed to affect uniform packing of the fibers 20. For example, the tubing can be formed of a polymeric composition including e.g., polyethylene (PE), and the resulting column 22 then can be surrounded by a water jacket. Increases in pressure applied to the jacket can squeeze the polyethylene column 22 and thus compress the fibers 20 into a tighter bundle.

Chemical separations of model compound classes can be performed to assess the role of compression on the retention qualities of the different fibers. Particular attention is paid to the trade-offs between packing density, the obtained resolution, and the backing pressure required to provide the desired flow through the column 22.

According to the disclosure, a surface of the polymeric support phase can be modified to include a crosslinked polymer stationary phase. For instance, when considering a surface-channeled polymer fiber support phase, the support phase can be modified while maintaining the high surface area-to-volume ratio and the basic channeled structure. For instance, at least portions of the surface of channeled surfaces of the polymer fibers 20 can be modified to a predetermined chemical reactivity through inclusion of a crosslinked polymer network at the surface as a stationary phase.

The crosslinked polymer stationary phase can include a polymer that comprises reactive functionality for crosslinking as well as reactive functionality for interaction with an analyte of interest. Interaction with an analyte can be carried out according to covalent bonding, non-covalent bonding, ionic bonding, charge-charge interaction, or any other sort of reversible or irreversible interaction, as desired. For example, a polymer can include one or more of amine-, carboxylate-, hydroxyl-, thiol-functionality, as well as combinations of functionality. In one embodiment, a polymer can include imine functionality. For example, a support phase can be processed to include crosslinked polyethyleneimine) at a surface, for instance for use in ion-exchange chromatography in separations of biological molecules such as proteins.

As is known, ion-exchange chromatography (IEC) is a widely used technique in bio-separations, including protein downstream processing, because of the use of solvent conditions that do not affect large amounts of protein denaturation. Bio-molecules have ionizable chemical moieties that can be used to retain the molecule on the charged IEC stationary phase. When the environmental conditions are changed (e.g., pH, ionic strength), the retained bio-molecules can then be sequentially eluted from the stationary phase to realize the separation.

The amine-rich polymer, polyethyleneimine (PEI) is attractive for use in IEC separation due to low cost and availability. Moreover, PEI contains primary amines, as well as secondary amines and tertiary amines, which have better ion-exchange capacity than primary amines. In a PEI modification, the polyamines can react with multiple ester bonds of the PET as schematically illustrated in FIG. 5. As such, the broken ester bonds can be crosslinked by the polymeric structure of PEI. As a result, the mechanical strength loss from the PEI modification can be minimum or negligible. A polymer such as PEI can also be excluded from any pore structure of the support phase, as well as from the fiber-free volume. As such, the polymer modification can occur only on the surfaces of the support phase, resulting in ion-exchangers with better accessibility for the targeted species, e.g., proteins.

The polymer can be crosslinked by use of any suitable crosslinking agent that can crosslink the polymer without destroying the desired interactivity capability of the polymer with the analyte of interest. The crosslinking agent can include a polyfunctional compound that can react with functionality of the polymer to form crosslinks within and among the polymer chains on the surface of the support phase. In general, the crosslinking agent can be a non-polymeric compound, i.e., a molecular compound that includes two or more reactively functional terminal moieties linked by a bond or a non-polymeric (non-repeating) linking component. By way of example, the crosslinking agent can include but is not limited to di-epoxides, poly-functional epoxides, diisocyanates, polyisocyanates, polyhydric alcohols, water-soluble carbodiimides, diamines, diaminoalkanes, polyfunctional carboxylic acids, diacid halides, polyglycidyl ethers, such as ethylene glycol diglycidyl ether, 1,4-butane diglycidyl ether, and polyethylene glycol dicglycidyl ether; acrylamides; compounds containing one or more hydrolyzable groups, such as alkoxy groups (e.g., methoxy, ethoxy and propoxy); alkoxyalkoxy groups (e.g., methoxyethoxy, ethoxyethoxy and methoxypropoxy); acyloxy groups (e.g., acetoxy and octanoyloxy); ketoxime groups (e.g., dimethylketoxime, methylketoxime and methylethylketoxime); alkenyloxy groups (e.g., vinyloxy, isopropenyloxy, and 1-ethyl-2-methylvinyloxy); amino groups (e.g., dimethylamino, diethylamino and butylamino); aminoxy groups (e.g., dimethylaminoxy and diethylaminoxy); and amide groups (e.g., N-methylacetamide and N-ethylacetamide).

The surface modification to include the crosslinked separation phase on the polymeric support phase can be carried out at any point, for instance prior to formation of a separation device (e.g., on individual fibers prior to packing in a column) or in situ following formation of the separation device.

The crosslinked network may contain “intra-cross links” (i.e., covalent bonds between functional groups of a single molecule) and/or “inter-cross links” (i.e., covalent bonds between different molecules, e.g., between two polymers of the network or between a polymer and the surface of the support phase). Crosslinking may be carried out via self-crosslinking of the indicator and/or through the inclusion of a separate crosslinking agent.

Any of a variety of different crosslinking mechanisms may be employed, such as thermal initiation (e.g., condensation reactions, addition reactions, etc.), electromagnetic radiation, and so forth. Some suitable examples of electromagnetic radiation that may be used include, but are not limited to, electron beam radiation, natural and artificial radio isotopes (e.g., α, β, and γ rays), x-rays, neutron beams, positively-charged beams, laser beams, ultraviolet, etc. Electron beam radiation, for instance, involves the production of accelerated electrons by an electron beam device. Electron beam devices are generally well known in the art. The wavelength λ of the radiation may vary for different types of radiation of the electromagnetic radiation spectrum, such as from about 10-14 meters to about 10-5 meters. Electron beam radiation, for instance, has a wavelength λ of from about 10-13 meters to about 10-9 meters. Besides selecting the particular wavelength λ of the electromagnetic radiation, other parameters may also be selected to control the degree of crosslinking. For example, the dosage may range from about 0.1 megarads (Mrads) to about 10 Mrads, and in some embodiments, from about 1 Mrads to about 5 Mrads.

Initiators may be employed in some embodiments that enhance the functionality of the selected crosslinking technique. Thermal initiators, for instance, may be employed in certain embodiments, such as azo, peroxide, persulfate, and redox initiators. Representative examples of suitable thermal initiators include azo initiators such as 2,2′-azobis(2,4-dimethylvaleronitrile), 2,2′-azobis(isobutyronitrile), 2,2′-azobis-2-methylbutyronitrile, 1,1′-azobis(1-cyclohexanecarbonitrile), 2,2′-azobis(methyl isobutyrate), 2,2′-azobis(2-amidinopropane)dihydrochloride, and 2,2′-azobis(4-methoxy-2,4-dimethylvaleronitrile); peroxide initiators such as benzoyl peroxide, acetyl peroxide, lauroyl peroxide, decanoyl peroxide, dicetyl peroxydicarbonate, di(4-t-butylcyclohexyl)peroxydicarbonate, di(2-ethylhexyl)peroxydicarbonate, t-butylperoxypivalate, t-butylperoxy-2-ethylhexanoate, and dicumyl peroxide; persulfate initiators such as potassium persulfate, sodium persulfate, and ammonium persulfate; redox (oxidation-reduction) initiators such as combinations of the above persulfate initiators with reducing agents such as sodium metabisulfite and sodium bisulfite, systems based on organic peroxides and tertiary amines, and systems based on organic hydroperoxides and transition metals; other initiators such as pinacols; and the like (and mixtures thereof).

Photoinitiators may likewise be employed, such as substituted acetophenones, such as benzyl dimethyl ketal and 1-hydroxycyclohexyl phenyl ketone; substituted alpha-ketols, such as 2-methyl-2-hydroxypropiophenone; benzoin ethers, such as benzoin methyl ether and benzoin isopropyl ether; substituted benzoin ethers, such as anisoin methyl ether; aromatic sulfonyl chlorides; photoactive oximes; and so forth (and mixtures thereof). Other suitable photoinitiators may be described in U.S. Pat. No. 6,486,227 to Nohr, et al. and U.S. Pat. No. 6,780,896 to MacDonald, et al., both of which are incorporated herein by reference.

Although not required, additional components may also be employed within the crosslinked network to facilitate the securement of the stationary phase. For example, an anchoring compound may be employed that links the stationary phase polymer to the surface of the support phase. For example, the anchoring compound may include a macromolecular compound, such as a polymer, oligomer, dendrimer, etc. Polymeric anchoring compounds may be natural, synthetic, or combinations thereof. Examples of synthetic polymeric anchoring compounds include, for instance, polyacrylic acid and polyvinyl alcohols.

Of course, anchoring compounds are not required in the disclosed systems. For instance, in some embodiments, the support phase and the separation phase can bond one another directly. By way of example, a polyester support phase such as PEI can directly bond an amine-containing polymer separation phase polymer such as PEI. Due to the large molecular size of PEI, the primary amines can react with the esters on PET surfaces without entering the bulk structure, minimizing physical integrity loss of the fibers while retaining additional amine reactivity for crosslinking and interaction with the targeted species.

When used, the manner in which an anchoring compound is used to link the polymer and the support phase may vary. In one embodiment, for instance, the anchoring compound is attached to the polymer prior to application of both to the support phase. In other embodiments, the anchoring compound may be bonded to the support phase prior to application of the polymer. In still other embodiments, the materials may be applied as separate components to the support phase and attachment reactions can take place in situ, optionally at the same time as the crosslinking of the network. For instance, the polymer may bind the anchoring compound, the anchoring compound may bind the support phase, and simultaneously, cross-linking reactions can take place between anchoring compounds, between polymers, or between the two.

In the case of bonds being formed between the system components, attachment of an anchoring compound to a support phase as well as attachment of the anchoring compound to the polymer may be accomplished using carboxylic, amino, aldehyde, bromoacetyl, iodoacetyl, thiol, epoxy or other reactive functional groups, as well as residual free radicals and radical cations, through which a binding reaction may be accomplished and according to any suitable methods, e.g., thermal processes, photo-initiated processes, catalyzed reactions, and the like. For example, a support phase may be amine-functionalized through contact with an amine-containing compound, such as 3-aminopropyltriethoxy silane, to increase the amine functionality of the surface and bind the anchoring compound and/or the polymer to the surface via, e.g., aldehyde functionality of the component.

It should be understood that, besides covalent bonding, other attachment techniques, such as charge-charge (ionic) interactions, may also be utilized for attaching the anchoring compound to the support phase and/or for attaching the polymer to the support phase or the anchoring compound. For instance, a charged anchoring compound, such as a positively charged polyelectrolyte anchoring compound, may be immobilized on a negatively charged support phase through charge-charge interactions between the two.

In addition, it should be understood that the disclosure is not limited to attachment of a single crosslinked polymer to a support phase. For instance, analyte interactive functionality density may be increased through binding of a plurality of crosslinked polymers to a single support phase. The different polymers can be combined into a single composite network or in separate areas of the support phase. In one embodiment, multiple crosslink layers can be formed on the support phase. For instance, following formation of a first crosslinked layer, a second crosslinked layer of the same or a different polymer can be formed on top of the first layer so as to increase the surface area and species interaction sight density of the separation phase. Each layer can be crosslinked prior to the formation of a next layer or alternatively, following addition of multiple layers, the layers can be crosslinked in a single crosslink step.

Once the polymer layer has been applied, crosslink reactions may occur to form the crosslinked network. Depending upon the nature of the materials of any specific embodiment, crosslink reactions may occur between two of the support phase, the polymer, and, when present, the anchoring compound as well as between any two of the same components as inter-crosslinks (e.g., between two anchoring compounds or between two polymers) or among a single component as intra-crosslinks (e.g., between two functional moieties on a single polymer). For example, an anchoring compound and a polymer may be applied as a mixture to the support phase, optionally with a suitable crosslinking agent and/or a crosslink initiator. Upon initiation of the binding reactions (e.g. photoinitiation), a crosslinked network may be formed that includes the crosslinked polymer at the surface of the support phase.

Any suitable binding mechanism may be employed to facilitate crosslinking. By way of illustration only, examples of compounds that may be cured via a photoinitiated crosslinking process may include those including unsaturated monomeric or oligomeric groups such as, without limitation, ethylene, propylene, vinyl chloride, isobutylene, styrene, isoprene, acrylonitrile, acrylic acid, methacrylic acid, ethyl acrylate, methyl methacrylate, vinyl acrylate, allyl methacrylate, tripropylene glycol diacrylate, trimethylol propane ethoxylate acrylate, epoxy acrylates, such as the reaction product of a bisphenol A epoxide with acrylic acid; polyether acrylates, such as the reaction product of acrylic acid with an adipic acid/hexanediol-based polyether, urethane acrylates, such as the reaction product of hydroxypropyl acrylate with diphenylmethane-4,4′-diisocyanate, and polybutadiene diacrylate oligomer.

Other surface modifications can be carried out in addition to the inclusion of the crosslinked polymer as stationary phase on the support phase. For example, in one embodiment, a predetermined chemical reactivity can be obtained by modifying at least portions of the surfaces of a polymeric support phase to a predetermined level of hydrophobicity. Thus, active sites on the fiber surfaces can be functionalized to gain more or less hydrophobic character. A predetermined chemical reactivity also can be obtained by modifying at least portions of the surface of the support phase to a predetermined ionic character. For example, the surfaces of fibers 20 formed from polyvinyl alcohol (PVA) might be protonated in situ by an acidic mobile. Such additional modification can be carried out in the same surface location of the support phase as at the crosslinked polymer or in a different area, as desired. For instance a first area of a support phase can be modified to exhibit a predetermined hydrophobic character and a second area of the support phase can be modified to include the crosslinked polymer to exhibit a predetermined ionic character. Alternatively, both modifications can be carried out over the same surface of the support phase. For instance, following modification of a support phase to include a crosslinked polymer, further modification can be carried out to modify the crosslinked polymeric network to include, e.g., a particular hydrophobicity, a particular functional group, or the like.

While a preferred embodiment of the invention has been described using specific terms, such description is for illustrative purposes only, and it is to be understood that changes and variations may be made without departing from the spirit or scope of the following claims. Specifically, it should be understood that the chemistries involved to be implemented on other support phases having the proper inherent surface chemistries, to which the described stationary phase could be affixed. In any case, the chemistries could be affected on the support phases in a bulk fashion, followed by assembly in a column format or perhaps performed in situ once the column has been packed with the support phase.

The present invention may be better understood with reference to the Examples set for below.

### Example 1

Unless otherwise specified, chemicals were purchased from commercially available sources and used without further purification. Polyethylenimine (PEI, MW 10,000, 99%) was purchased from Polysciences, Inc. (Warrington, Pa.). Dimethyl sulfoxide (DMSO, ACS grade), pyridine (99%), Tris base (99.8%) were purchased from VWR (Atlanta, Ga.). 1,4-Butanediol diglycidyl ether (BUDGE, 95%) and all proteins were purchased from Sigma-Aldrich (St. Louis, Mo.). All HPLC solvents were purchased from EMD (Billerica, Mass.). Deionized water (DI—H₂O) was obtained from a Milli-Q water system.

PET capillary-channeled fibers were obtained from the Clemson University School of Materials Science and Engineering. 450 PET fibers were pulled through polyether ether ketone tubing (PEEK, 0.762 mm i.d., IDEX Health & Science LLC, Oak Harbor, Wash.). After packing, the columns were mounted on a Dionex Ultimate 3000 HPLC system (LPG-3400SD Quaternary pump, MWD-3000 UV-vis absorbance detector, Thermo Fisher Scientific Inc., Sunnyvale, Calif.) and washed with acetonitrile then deionized water until a stable baseline was observed at 216 nm. Once assembled and cleaned, the microbore columns could be stored in ambient conditions and cut to appropriate lengths prior to surface modifications.

The fibers were packed in columns before modification to include a crosslinked PEI network on the surface. Before modifications, the columns were mounted on a HPLC pump and washed with DMSO at 0.5 mL min⁻¹ for 10 min. The columns were then placed in a column heater assembly and connected to a syringe pump that contained the reactive PEI solution. The column heater was set to 100° C., and solutions of 15% PEI in DMSO were continuously pumped through the columns at a flow rate of 0.6 mL h⁻¹ for 2 h, 3 h or 4 h. As such, the surface modification chemistries were varied from 1 to 4 h. The resulting columns were designated as PET-PEI-2 h, PET-PEI-3 h, and PET-PEI-4 h. After the modifications, the columns were brought up to ambient temperature and mounted on the HPLC to be washed with DMSO and water until a stable baseline at 216 nm was observed; reflective of removal of unreacted/unbound PEI.

Another set of columns were modified to include a crosslinked PEI network on the fibers. Before modifications columns were mounted on a HPLC pump and washed with DMSO at 0.5 mL min⁻¹ for 5 min. Different from the simple modification described above, the inclusion of BUDGE crosslinking and multiple repetitions were undertaken under static conditions. In the initial PEI modification step, 10 column volumes (CV) of 15% PEI in DMSO were pumped through the column. Then, the columns were sealed at both ends and placed in an oven at 100° C. for 20 min. After the initial PEI modification, the columns were cooled to room temperature and washed with DMSO at 0.5 mL min⁻¹ for 5 min. For the BUDGE modification, 10 CV of 15% BUDGE in DMSO were pumped through the column, the columns were re-sealed, and placed in an oven at 100° C. for 20 min. The columns were washed with DMSO at 0.5 mL min⁻¹ for 5 min between each modification to remove the unreacted PEI or BUDGE.

For the column designated PET-PEI/BUDGE #1, the column was first modified by PEI, followed by BUDGE cross-linking, and an additional PEI application. For the column designated PET-PEI/BUDGE #2, BUDGE and PEI modifications were repeated once beyond that of PET-PEI/BUDGE #1. Finally, for the column designated PET-PEI/BUDGE #3, the BUDGE and PEI modifications were repeated twice beyond that of PET-PEI/BUDGE #1. After each modification procedure was completed, the columns were mounted on the HPLC and washed with DMSO and water until a stable baseline absorbance values at 216 nm were observed. Columns were stored under ambient condition with a DI—H₂O fill and sealed with end caps.

The ninhydrin test method was used to determine the primary and secondary amine density on the fiber surfaces. Each fiber sample (5-6 mg) was placed into a test tube containing 0.5 mL of 10% isopropanol, 0.5 mL of 10% pyridine and 1 mL of the ninhydrin solution composed of 2.325 mL propionic acid, 12.5 mL 2-methoxyethanol, 5.045 g sodium propionate, and 0.5 g ninhydrin, with the total volume adjusted to 25 mL with deionized water. The test tubes were sealed and heated in a boiling water bath for 30 min. After removal and cooling, 10 mL of 50% ethanol was added to each test tube. The tube was shaken and then left to sit for 15 min. The mixture solution in each test tube was diluted to 25 mL with deionized water prior to the absorbance measurement. The PEI solutions with known concentrations were analyzed following the same procedure and used as calibrants. A ninhydrin test containing no fibers or PEI was used as the blank. The concentration of the PEI was quantified by the absorbance measurements of diluted ninhydrin test solutions performed at 570 nm. A field emission scanning electron microscope (Hitachi, SU6600, Japan) was used to examine the microscopic morphology of the fibers. The SEM was set to Variable Pressure (VP) mode at 30 Pa. The accelerating voltage was set to 15 kV. An atomic force microscope (AIST-NT Smart SPM 1000, AIST-NT, USA) operating in the AC mode was used to examine the surface morphology of the fibers. The AIST-NT Control software was used to generate the AFM images. The Plane Level function in the software was applied to all AFM images.

All chromatography experiments were performed on a Dionex Ultimate 3000 HPLC system. The column hydrodynamic properties were determined using DI-H₂O as the mobile phase at different flow rates. The column permeability was calculated by Eq. (1):

$$\frac{Δ P}{L} = \frac{u μ}{{k}_{w}} \qquad ( 1 )$$

where ΔP represents the column backpressure (Pa),
L represents the column length (m),
u represents the linear velocity of the mobile phase (m s⁻¹),
μ represents the mobile phase viscosity (Pa s) and
k_{w} represents the column hydraulic permeability.

The dynamic binding capacity (DBC) of the column was determined by load/elution experiments using BSA as the model protein. After equilibration with buffer A (20 mM Tris-HCl buffer, pH7.5), the column was loaded with different concentrations of BSA in buffer A at a constant flow rate until a consistent absorbance value for the eluate was achieved, reflecting column saturation. Buffer A was then applied to the column until the absorbance at 280 nm of the eluent returned to baseline, effectively indicating that no non-bound protein existed in the column. Buffer B (1 M NaCl in buffer A) was then applied to the column for BSA elution. The DBC was calculated based on the integrated absorbance value elution peak as compared to calibration curves prepared from injections of BSA without a column present in the system. The response curves were generated with BSA dissolved in 90:10 acetonitrile (ACN):H2O (RP solvent) and 1 M NaCl in Tris buffer (AEC solvent).

The relative chromatographic performance of the columns was evaluated by injection of a three-protein mixture (BSA, hemoglobin and lysozyme) at different flow rates, with the same gradient programs/rates of buffer A and B. The chromatograms were recorded as the optical absorbance at 280 nm.

In these experiments, excess amounts of BUDGE were applied in each step, with the unreacted BUDGE in the solution phase washed away. Any partially-reacted BUDGE, with an unreacted epoxide group, remained on the PET fiber surfaces (FIG. 5). When a subsequent PEI modification was applied, the PEI reacted with the remnant epoxide group, increasing the PEI density on the PET surfaces. As illustrated in FIG. 5, the PEI-BUDGE reaction cycles can be repeated multiple times.

The ninhydrin reaction is an effective means to quantify primary and secondary amines in solution and on fiber surfaces. While the most desirable form of amine for anion exchange chromatography (AEX) separations are tertiary amines, these are not quantifiable. Based on the assumption that the 1°/2°/3° ratio in PEI was 25/50/25 (provided by the provider), the ninhydrin test was expected to provide a reflection of the 3° species (though this value is still an under estimate as the BUDGE treatment favors an over production of tertiary species versus the other forms). The results of the ninhydrin determinations are presented in Table 1 as a function of the surface modification conditions.

TABLE 1

| | 1°/2° amine | 1°/2° amine | | | |
|---|---|---|---|---|---|
| | density | density | Permeability | Qₘ | |
| Stationary phases | (μmol g⁻¹)^{a} | (μmol m⁻²)^{a, b} | (10⁻¹¹ m²) | (mg ml⁻³)^{c} | R³ |
| Native PET | ND | ND | 1.35 | 0.41 | 0.9630 |
| PET-PEI-2 h | 74.8 ± 5.2 | 24.9 ± 1.7 | 1.33 | 1.99 | 0.9887 |
| PET-PEI-3 h | 97.4 ± 14.9 | 32.5 ± 4.6 | 1.11 | 2.28 | 0.9363 |
| PET-PEI-4 h | 142.7 ± 8.7 | 47.6 ± 2.9 | 0.98 | 2.35 | 0.9843 |
| PET-PEI/BUDGE #1 | 111.4 ± 5.2 | 37.1 ± 1.7 | 1.05 | 7.15 | 0.9791 |
| PET-PEI/BUDGE #2 | 135.7 ± 10.4 | 45.2 ± 3.5 | 0.79 | 8.54 | 0.9852 |
| PET-PEI BUDGE #3 | 154.9 ± 17.4 | 51.6 ± 5.8 | 0.66 | 8.32 | 0.9814 |

ND: not detected.
^{a}Molar concentration based on PEI molecular weight of 10,000 Da.
^{b}Areal density based on fiber specific surface area of 3 m²g⁻¹.
^{c}Based on fitting of breakthrough data to a simple Langmuir isotherm.

As would be expected, increased reaction times for the straight PEI treatments result in higher densities of 1° and 2° amines, though the effect is not linear across the intermediate (3 h) reaction time. This would seem to suggest that the formation of the PEI layer on PET does not follow a simple kinetic mode. The upper density value was about ˜143 μmol g⁻¹ (48 μmol m⁻²).

When considering the alternating PEI-BUDGE treatments, one would expect that the sequential layers would indeed provide greater primary and secondary amine densities, though with the caveat that there should be some disproportion toward tertiary amines as noted above. The results presented in Table 1 suggest that the initial step did yield greater ninhydrin accessible 1°/2° amines, but the successive treatments do not yield appreciable greater densities than the extended period treatments with neat PEI. The PEI density differences indicate that the PEI attachment efficiency of the PEI-BUDGE double modification is higher than the PEI single modification, which is attributed to the BUDGE crosslinking. Each additional PEI-BUDGE cycle added an equivalent amount of detectable amine density. While seemingly a more complex process, the sequential modification cycles occur over a time frame of 80 min for the PEI-PEI/BUDGE#3, whereas the longest neat PEI treatment takes 4 h. It would seem reasonable, based on the chemistry taking place in the course of the BUDGE cross-linking step, that this process should result in a higher density of tertiary amines than the simple PEI treatment of PET, which should be reflected in higher dynamic binding capacities.

Cutting of the columns into thirds and performance of the ninhydrin test of the individual segments showed quite good homogeneity for the PET-PEI-4 h and PET-PEI/BUDGE #2 columns. Triplicate measurements for the PEI-modified column showed no deviation in the 1°/2° amine content along the column length outside of the measurement precision of ˜6% RSD. In the case of the PEI/BUDGE modification, the column head and middle portion values were statistically the same, while there was a small discernible decrease (˜9%) at the column end. This lesser homogeneity (albeit relatively small) was likely the result of the shorter exposure times in the sequential steps of the PEI/BUDGE processing.

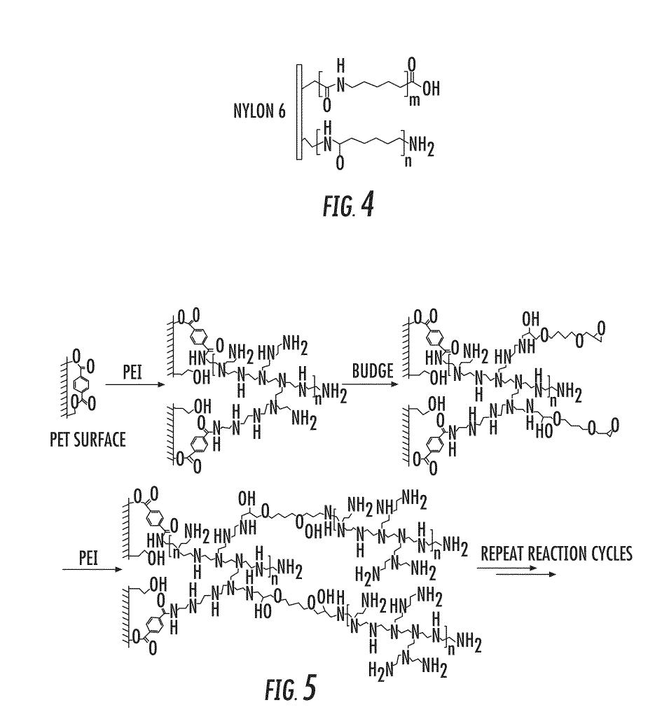

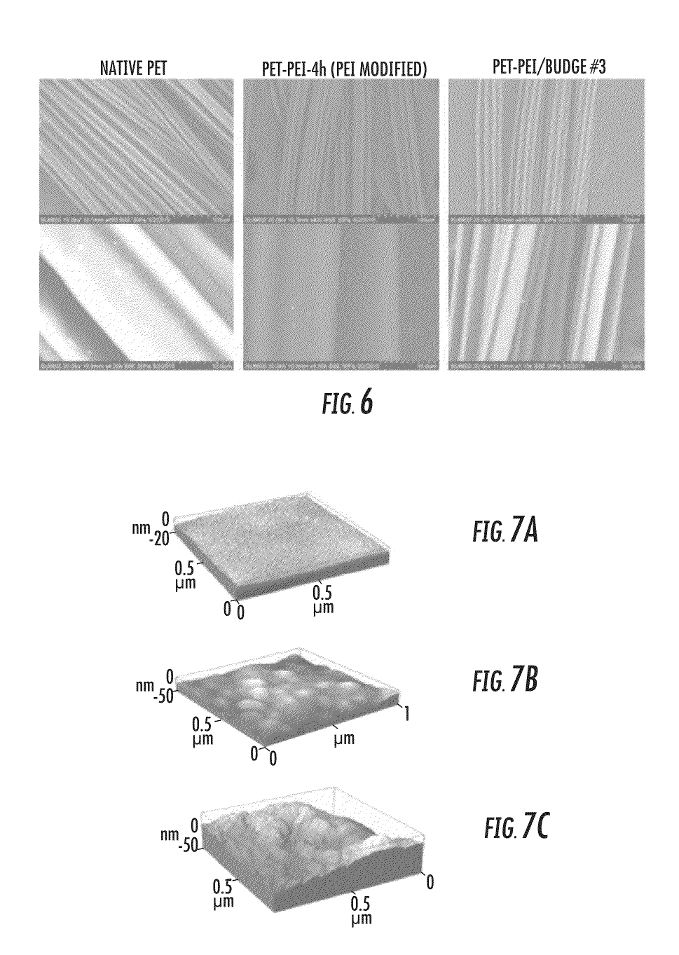

FIG. 6 shows SEM images of the native PET, and the PET-PEI-4 h and PET-PEI/BUDGE #3 fibers that have recovered from the columns following chemical processing. The channel structure appears to be unperturbed following the surface modifications on the micrometer scale of the fiber channels. In addition, there did not appear to be any deformation of the channels due to the 100° C. processing temperatures. To further examine the potential surface morphology changes caused by the modifications, the fibers were probed via AFM. As shown in FIG. 7A, FIG. 7B, FIG. 7C, the nanometer-scale vertical resolution of AFM revealed substantial changes on the 1 μm² fiber plane. Without any modification (FIG. 7A), the native PET fiber surface was seen to be relatively smooth and flat, with the variations of the surface features being ˜±0.5 nm (the 20 nm vertical scale in that figure is due to a tilt of the fiber surface on the AFM stage).

The surfaces of the PEI- and PEI-BUDGE-modified surfaces (FIG. 7B and FIG. 7C respectively) show dramatically increased surface topography than the PET fibers. In the case of the PEI-modified fiber broad features of 1-3 nm heights were seen, while in the case of the PEI-BUDGE-modified PET fiber much larger features were created. It is clear from these images that polymer layers have been created on the fiber surfaces, with the PEI-BUDGE apparently generating a much thicker, topographically structured layer.

FIG. 8 depicts the responses of back pressures of the PET fiber packed columns at different mobile phase linear velocities. The good linear relationship between the column backpressures and the mobile phase linear velocities indicates negligible fiber compression or other bed perturbation across the linear velocity range of about 44-526 cm min⁻¹ (equating to volume flow rates of 0.1-1.2 mL min⁻¹). In comparison to the native PET column, the increasing slopes of the responses reflect decreases in permeability for all PEI or PEI-BUDGE modified columns (Table 1). In the PEI modified columns, the columns with longer modification time have lower permeability, which would reflect thicker polymer coatings. In the PEI-BUDGE modified columns, the columns with more reaction cycles have lower permeability, as would be expected. In general, the on-column modifications did not appear to appreciably degrade the hydrodynamic characteristics of the channeled fiber columns, as high throughput could be achieved at pressures easily maintained by standard HPLC systems.

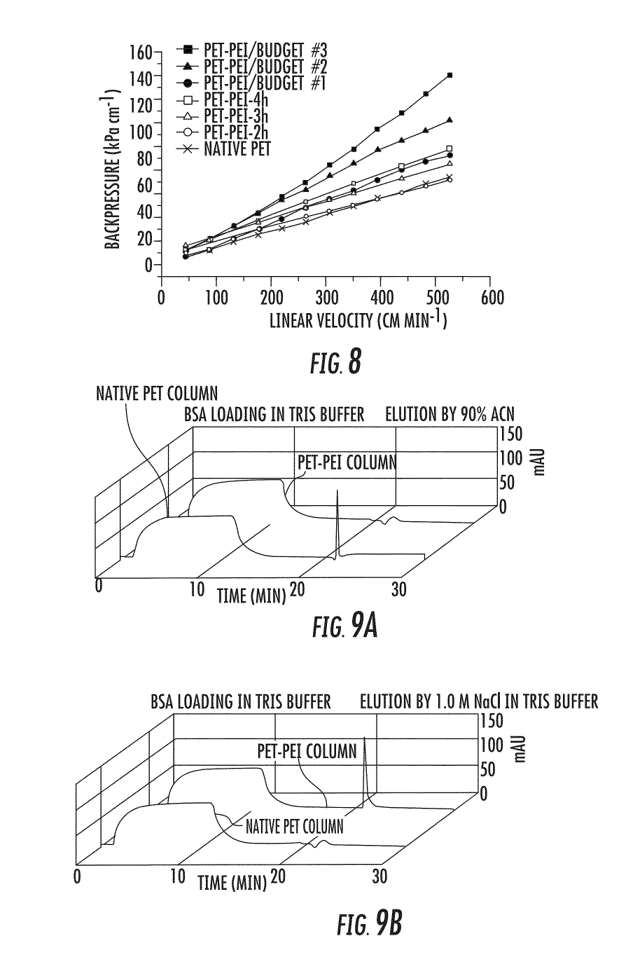

BSA was used as the model protein for chromatography studies. FIG. 9A and FIG. 9B show the protein load/elution transients under reversed phase and ion-exchange elution conditions for the native PET and a PET-PEI-4 h column. BSA (1 mg mL⁻¹) in Tris-HCl buffer was loaded on each column until saturation was achieved. The protein was then eluted with 90:10 ACN:H₂O (RP) (FIG. 9A) or 1 M NaCl in Tris-HCl buffer (pH 7.5) (AEC) (FIG. 9B). Under reversed phase conditions, the native PET column yielded a detectable elution peak, while no elution was observed from the PEI-modified PET column. Under ion-exchange elution conditions, the PEI-modified PET column yields a clear elution peak, while no elution peak was observed from the native PET column. The other PEI-BUDGE modified PET fiber columns behave in the same manner as the example here. This relatively simple experiment clearly showed that the BSA interactions with native PET are predominately hydrophobic in nature, with the interactions with the PEI phase being ionic in nature. The protein binding capacity of the modified PET fiber columns was determined through dynamic load/elution experiments and creation of isotherms.

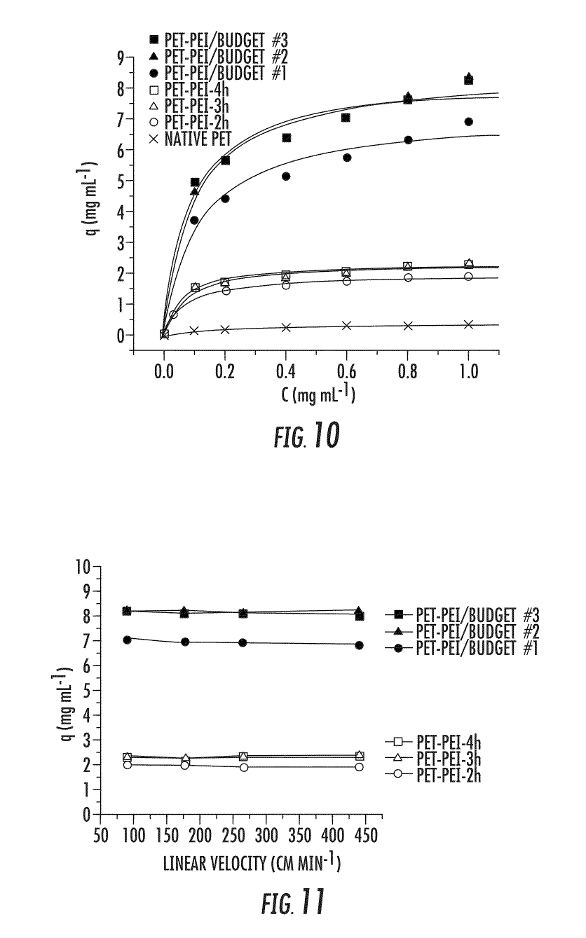

BSA (0.1-1 mg mL⁻¹) in Tris-HCl buffer (20 mM, pH 7.5) was loaded onto columns at a flow rate of 1 mL min⁻¹ (438 cm min⁻¹) until saturation was achieved as judged by the measured absorbance at 280 nm. The columns were then washed with Tris-HCl buffer until the absorbance at 280 nm returned to baseline values. The elution buffer (1 M NaCl in 20 mM Tris-HCl, pH 7.5) was introduced onto the column until complete elution was achieved. The amount of protein retained on the columns was calculated based on the integrated absorbance of the elution peak and comparisons to calibration curve responses. FIG. 10 depicts the dynamic loading characteristics for the native PET and different PEI modification conditions, as a function of loading BSA concentrations (analogous to the measured equilibrium concentration in a static loading experiment). The data were fit to the Langmuir model (Eq. (2)),

$$q = \frac{{Q}_{m} c}{{K}_{d} + c} \qquad ( 2 )$$

where c refers to the load concentration of the protein,
q refers to the loading capacity of the column at the protein concentration c,
Qₘ refers to the maximum DBC at infinite protein loading concentration and
K_{d} refers to the dissociation constant.

As can be seen, the respective data sets for the different modification conditions are well approximated by the Langmuir fits. Also clear, the data fit into three distinct groups; the native PET, neat PEI-modified PET, and the sequential PEI-BUDGE treatments. The Qₘ values that were obtained from the Langmuir equations are listed in Table 1. The DBC of the native PET channeled fibers was not detectable under IEC elution conditions as suggested from the responses in FIG. 9A, 9B, and so its value represents the same injection conditions but with the RP elution solvent. The 0.41 mg mL⁻¹ binding capacity value here is not unreasonable versus the DBC for BSA on nylon 6 channeled fibers of 0.31 mg mL⁻¹ obtained via frontal analysis of breakthrough curves. This value is considered to be the DBC baseline based on the binding kinetics (flow rate) and formation of a monolayer of protein on the available fiber surface. Clearly, a dramatic increase in dynamic binding capacity is realized with the simple PEI modification, with the Qₘ values increasing with modification time. The ˜5× increase in binding capacity upon the initial PEI modification clearly points to the improved enthalpic interactions between the BSA and the aminated surface versus the native PET. Based on the relative permeability of these two columns, there would appear to be no appreciable differences in the stationary phase surface areas.

What is seen here is dramatically enhanced capacity due to the presence of tertiary amines in the PEI case. It is not surprising that the extent of the Qₘ value increases for PEI-modified PET columns do not follow the ˜2× increase seen in the results of the ninhydrin tests as a function of PEI reaction time. Increased PEI layer thickness is reflected in the lower column permeabilities with reaction time. The ninhydrin results reflect the ability of the small molecule to permeate into the polymer layer, whereas there will not likely be substantial protein ingress. Likewise, there is no guarantee that the number of tertiary amines increases in direct proportion to the primary and secondary species. Ultimately, the relatively straightforward chemistries here provide substantial increases in dynamic binding capacity under AEX elution conditions. When BUDGE crosslinking was used in the PEI modification process, the Qₘ values of the columns dramatically increase. The Qₘ values of the PEI-BUDGE double modified PET columns are about 4 times greater than the neat-PEI modified columns. Clearly reflected in the AFM micrographs of FIG. 7A-7C and the permeability values of Table 1, the thickness and surface area of the polymer films are far greater for the PEI-BUDGE system, even though the overall reaction times are far less for the BUDGE system. In comparison, the ninhydrin-based 1°/2° amine densities are not appreciably different in the two cases of the most extensive modifications. As noted previously, the BUDGE modification should yield a higher fraction of 3° amines than the 25:50:25 distribution expected for PEI. Thus it is concluded that the BUDGE modification provides greater capacity through the generation of high tertiary amine densities; that due to the observed topology changes are more accessible by the BSA solutes. This point is made most definitively in comparing the capacities of PET-PEI-4 h and PEI-PEI/BUDGE #1, where the former has ˜25% higher 1°/2° amine densities, comparable column permeabilities, and yet a factor of 3 lower BSA binding capacity.

There was no increase in the DBC values between the PEI-PEI/BUDGE #2 and #3 conditions, as might be expected based on the results of the ninhydrin test. In this instance, it is believed that there is limited protein ingress to the polymer layer, as well as perhaps constriction within the column structure as reflected in the reduced column permeability.

An advantage of using channeled fiber columns for protein separations is the low mass transfer resistance of the bed. FIG. 11 shows the DBCs of the modified PET channeled fiber columns at different feed rates for a BSA feed concentration of 1 mg mL⁻¹. By changing the flow rates from ˜88 cm min⁻¹ to ˜438 cm min⁻¹ (equivalent to volume flow rates of 0.2-1 mL min⁻¹ for these columns), there were no significant changes on the DBC values for any of the modified columns. The results suggest that fast protein separations can be achieved on the PEI-modified PET channeled fiber columns by increasing the flow rate without losing column capacity. The binding capacity of the modified PET channeled fiber media was determine at a very high linear velocity (˜438 cm min⁻¹).

The PET-PEI/BUDGE #2 column was tested for protein separations at different flow rates. FIG. 12 shows the chromatograms of separations of synthetic protein mixture containing lysozyme. Chromatograms for the AEX separation of lysozyme, hemoglobin and BSA (left to right) on PET-PEI/BUDGE #2 at four different flow rates. Separations were carried out with buffer A (20 mM Tris-HCl, pH 7.5) and buffer B (1 M NaCl in buffer A). The gradient was performed from 0% to 25% buffer B across the initial 0.25 mL volume, kept at 25% B for 1.75 mL, then buffer B was increased from 25% to 50% across a 0.25 mL volume and kept at 50% for a total of 2.0 mL hemoglobin, and BSA (0.3 mg mL⁻¹ for each protein), under the same gradient conditions. In this demonstration, the elution gradient program remained fixed as a function of elution volume across the various flow rates, rather than a time-based gradient. For each chromatogram, the linear velocity and gradient time are presented. On a constant volume basis, the chromatograms are virtually superimposable, even though the linear velocities are increased from 55 to 438 cm min⁻¹ (equivalent to volume flow rates of 0.125-1 mL min⁻¹). The only differences are the reduced absorbance values that come about due to the solute dilution with increasing flow rates. Based on the chromatograms of FIG. 12, the lack of significant change of peak shapes at increased flow rates implies that the observed peak broadening is neither due to the longitudinal diffusion (B-term in the van Deemter equation) or mass transfer resistance between stationary phase and mobile phase (C-term in the van Deemter equation). The peaks themselves, though, are broad and asymmetric. One might assume that surface diffusion on/within the polymer phase may be problematic, but this too would show up as a C-term phenomenon. Ultimately, it is the non-ideal packing of the column (van Deemter A-term) that results in the peak broadening which would not be a function of separation velocity. The broadening comes from variance in the intra-fiber spacing along the column. By the same token, the broadening seen here is most likely from non-homogeneous radial distribution of the flow velocities.

### Example 2

Four basic fiber/column systems are compared towards weak anion exchange (WAX) separations: native nylon 6, native PET, PEI-modified PET, and the PEI/BUDGE-modified PET.

Either 720 nylon 6 fibers or 450 PET channeled fibers were pulled through polyether ether ketone tubing (PEEK, 0.762 mm i.d., IDEX Health & Science LLC, Oak Harbor, Wash.) in such a way that the fibers were aligned in a parallel fashion, with column interstitial fractions of ≈0.55 as determined by the elution characteristics of proteins injected under nonretentive solvent conditions. After packing, the columns were connected to a Dionex Ultimate 3000 HPLC system (LPG-3400SD Quaternary pump and MWD-3000 UV-Vis absorbance detector, Thermo Fisher Scientific Inc., Sunnyvale, Calif.) and washed successively with acetonitrile and DI-H₂O until a stable baseline was observed at 216 nm. Once assembled and cleaned, the microbore columns were filled with DI—H₂O, capped, and stored at ambient conditions prior to surface modifications or protein separation.

PEI modification procedures were carried out as described in the Example above. PET columns were washed with DMSO at 0.5 mL min⁻¹ for 10 min, and then PEI modification was affected by passing a 15% (w/v) solution of PEI in DMSO through the columns at a flow rate of 0.6 mL h⁻¹ for 4 h at 100° C., using a column heater to control the column temperature. The resulting columns were designated as PET-PEI. After modification, the columns were equilibrated to ambient temperature and washed with ˜200 column volumes (CV) of DMSO. The columns were then connected to the HPLC to be washed with ACN and DI—H₂O to remove the unreacted chemicals on the fiber, until a stable absorbance baseline at 216 nm was observed in each case.

For the PEI/BUDGE double modification (two complete cycles), 10 CV of 15% PEI in DMSO were first pumped through the column. Then, the columns were sealed at both ends and placed in an oven at 100° C. for 20 min. After the initial PEI modification (and each of the subsequent treatments), the columns were cooled to ambient temperature and washed with DMSO at 0.5 mL min⁻¹ for 5 min. The initial crosslinking was accomplished by pumping 10 CV of 15% BUDGE in DMSO (v/v) through the column, and the columns were resealed and placed in an oven at 100° C. for 20 min. Unreacted PEI or BUDGE was removed by washing with DMSO at 0.5 mL min⁻¹ for 5 min. The BUDGE and PEI modifications were repeated to effectively create a double layer of crosslinked PEI. The columns were finally washed with ˜200 CV of DMSO and then connected to the HPLC and washed with ACN and DI-H₂O until a stable absorbance baseline was observed at 216 nm in each case. The resulting columns were designated as PET-PEI/BUDGE. All columns were stored under ambient conditions with a DI—H₂O fill and sealed with end caps.

All chromatography experiments were performed with absorbance detection at 280 nm. The relative chromatographic performance of the columns was evaluated by injection of a three-protein mixture (BSA, hemoglobin, and lysozyme) at different flow rates and gradient rates of mobile phase A (20 mM Tris-HCl buffer, pH=7.5) and B (1 M NaCl in mobile phase A). The chromatograms were recorded as the optical absorbance at 280 nm. After each separation, mobile phase A was applied to the column until the absorbance at 280 nm of the eluent returned to baseline, effectively indicating that no non-bound protein existed in the column.

Table 2 presents the essential characteristics of the fibers/columns.

TABLE 2

| | 1°/2° amine | | |
|---|---|---|---|
| Support/stationary | density | Permeability | Qₘ |
| phase | (μmol g⁻¹) | (10⁻¹¹ m²) [28] | (mg mL⁻¹)^{x} |
| Nylon 6 | 78.6 ± 1.2 [29] | 1.30 | 0.31^{b} [30] |
| Native PET | ND | 1.35 | 0.41 [28]^{b} |
| PET-PEI | 142.7 ± 8.7 [28] | 0.98 | 2.35 [28] |
| PEI-PEI/BUDGE | 135.7 ± 10.4 [28] | 0.79 | 8.54 [28] |

ND not detected
^{a}Qₘ is the maximum dynamic binding capacity at infinite protein loading concentration from fitting of breakthrough data to Langmuir isotherm
^{b}Based on reversed phase mode

In the first column, the molar densities of primary and secondary (1°/2°), as determined through ninhydrin reactions as described previously, are presented. As expected, no amines were detected on the native PET fibers. The initial PEI modification approximately doubled the density of the accessible amines over that of native nylon 6. The BUDGE modification did not appreciably shift the amine distribution toward more 2° and 3° species. In addition, there was no way to judge the fraction of amines that were actually accessible by the ninhydrin reagent.

The permeability of the columns is also presented. As shown, the native nylon 6 and PET columns had very similar permeability. Without consideration of the surface chemistry differences, they should have very similar protein loading characteristics. PEI and PEI/BUDGE modification of PET columns decreases the column permeability as the polymer layers are added to the fiber surfaces. It was expected that eluting proteins should change little in elution band widths based on the lower permeability.

The dynamic binding capacities of the respective columns were determined by dynamic loading-elution (recovery) studies, as described. A dramatic difference was seen between the native and modified fibers for the binding of BSA. In Table 2, the dynamic binding capacity for BSA is very similar on both the native nylon 6 and PET surfaces (under reversed phase conditions), while the PEI and PEI/BUDGE modifications increase the loadings appreciably, by approximately 20× in the latter case. This relationship seems reasonable if one assumes that proteins adsorb onto the surface in the native fibers, while the two modifications create greater surface area (as demonstrated in Example 1) may have some level of protein permeability within the PEI/BUDGE layers.

Single-protein injections were performed under a fixed set of chromatographic conditions. BSA solution (3 μL, 1.0 mg mL⁻¹ in Tris buffer) was injected onto each column under 100% of mobile phase A at a flow rate of 0.4 mL min⁻¹. The proteins were eluted with a 2-min gradient of 0 to 100% mobile phase B and the retention times/elution compositions recorded. As presented in Table 3, the BSA retention characteristics were statistically different for the three amine phases (single-protein injections were performed in triplicate on each column.

TABLE 3

| Support/stationary | | |
|---|---|---|
| phase | t_{R} (min) | % B |
| Native nylon | 2.21 ± 0.003 | 5.5 ± 2.5 |
| PET-PEI | 3.31 ± 0.006 | 60 ± 2.5 |
| PET-PEI/BUDGE | 3.14 ± 0.006 | 52 ± 2.5 |

Under the conditions of the separations, buffered at pH 7.5, the nylon 6 surface was zwitterionic, with approximately the same number of anionic and cationic sites, with the PEI-based phases being cationized amines. Seen was the fact that BSA experienced lesser amounts of electrostatic retention on nylon 6 than PEI. It is important to note that IEC is a displacement form of separation, and so the NaCl gradient can affect either cation or anion displacement with increasing salt content. Based on its isoelectric point of 4.7, BSA is charge-negative (net), but this does not explicitly mean that it is those species that are interacting with the stationary phase. In general, it would be expected that the interaction would be less extensive on the lower charge density (Table 2), zwitterionic surface.

The relative retentivity of BSA toward the PEI and PEI/BUDGE surfaces was similar but was lesser on the BUDGE crosslinked phase. In this case, while it was the PEI molecule that was the WAX-active component, there were two chemical differences that explain the retention characteristics. First, the crosslinking of the PEI entities shifted the distribution among the types of amines more toward secondary and tertiary forms, away from greater fractions of primary amines. In this way, the extent of the protein-amine interactions was reduced. A second contribution may be seen in the fact that the BUDGE crosslinking creates a more dense PEI architecture, which also contains some of the hydrophilic character of the epoxide linkers. Finally, if one considers that BUDGE is the linker of the ligand PEI, the relative retention follows previous observations that as ligand linker arms decrease in length, strong cation exchange is enhanced.

The protein recoveries were examined by injections of BSA and lysozyme as model proteins under non-retaining conditions. For each test, 3 μL of 1.0 mg mL⁻¹ BSA or lysozyme was injected in to a 20-cm column at 100% mobile phase B. The same injections were performed without columns installed and the elution peak was recorded as 100% recovery. The recoveries were determined by comparing to the elution peak areas with and without the column installed. The results were from the average of 5 injections and presented in Table 4. The BSA and lysozyme recoveries on the PET-PEI and PET-PEI/BUDGE columns range from 94 to 97%. On native nylon column, 94% recovery of BSA and 90% recovery of lysozyme were obtained.

TABLE 4

| Support/stationary | Recovery | Recovery |
|---|---|---|
| phase | of BSA (%) | of lysozyme (%) |
| Native nylon | 94 ± 2.1 | 90 ± 3.5 |
| PET-PEI | 96 ± 1.8 | 97 ± 1.4 |
| PET-PEI/BUDGE | 96 ± 1.4 | 94 ± 0.9 |

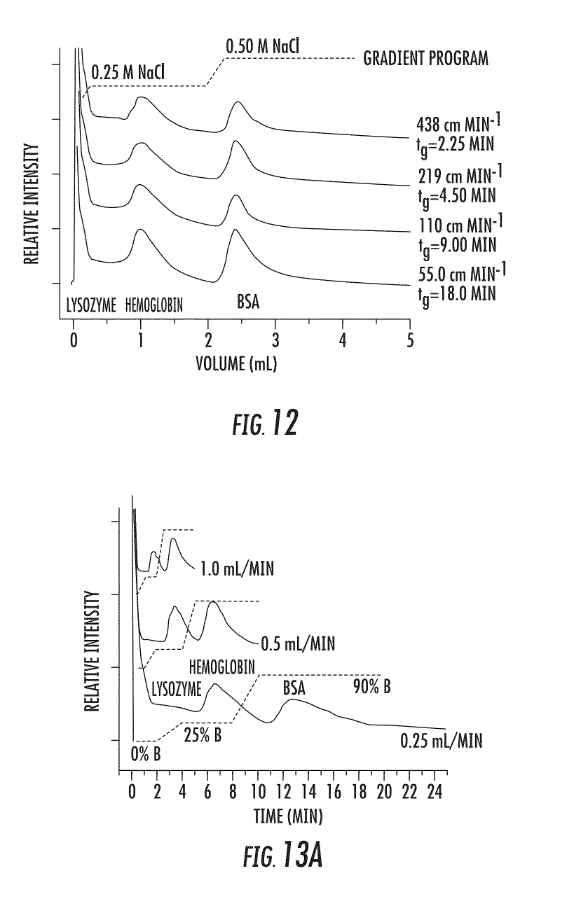

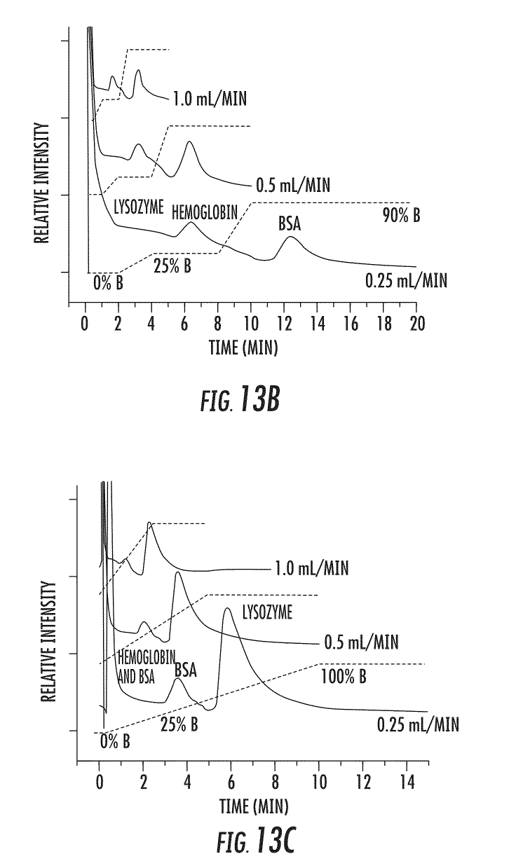

A synthetic three-protein mixture of BSA, lysozyme, and hemoglobin (0.25 mg mL⁻¹, each) was used to test the separation characteristics on PET-PEI, PET-PEI/BUDGE, and native nylon 6 C-CP fiber-packed columns. FIG. 13A, FIG. 13B, FIG. 13C show the elution profiles for 3-μL injections of the protein mixture at flowrates from 0.25 to 1.0 mL min⁻¹ (equivalent to linear velocities of 110-440 cm min⁻¹). In each separation, the gradient programs were kept the same on a volume basis, thus normalizing the gradient composition as a function of flow rate. Note that the gradient programs differ between the PEI-modified and native nylon fibers, with those employed being found as optimal for each phase. For each of the fiber types, the elution profiles were almost superimposable on the volume basis as the peaks each elute in the same nominal position in the gradient program. The predominate effect was a compression of the peak shapes on the time basis, as would be expected in the absence of mass transfer limitations. Essentially, fast protein separations could be realized on each of the columns by increasing flow rate, without sacrifice to the separation characteristics. The consistency reflects the low mass transfer resistance on channeled fibers as well as a lack of enthalpic changes that would be reflected in the % B of protein elution.

A comparison of the retention characteristics among the fiber types points to significant differences in the surface chemistries of PET-PEI, PET-PEI/BUDGE, and nylon 6. The retention times (and thus % B) of hemoglobin and BSA on PET-PEI/BUDGE were slightly shorter than those on PET-PEI. Lysozyme was not retained to an appreciable extent on either modified PET as it is charge-positive at this near-neutral pH, having a p/ of 11.35. Comparison of the chromatograms from PET-PEI (FIG. 13A) and PET-PEI/BUDGE (FIG. 13B) revealed that the eluting peaks of hemoglobin and BSA from the PET-PEI/BUDGE column were narrower (w_{1/2}=1.21 min for hemoglobin, w_{1/2}=1.57 min for BSA, at 0.25 mL min⁻¹) than the corresponding peaks from PET-PEI (w_{1/2}=2.45 min for hemoglobin, w_{1/2}=3.93 min for BSA, at 0.25 mL min⁻¹), which results in higher resolution (R=2.55 for PET-PEI/BUDGE, R=1.12 for PET-PEI, at 0.25 mL min⁻¹). In fact, the hemoglobin profile reveals the presence of a satellite peak, suggestive of a multiplicity of interactions. The peak broadening on PET-PEI column may be the result of the interaction between the protein and the base PET surface, which would be hydrophobic in nature. The increased hydrophobic interactions extend the range of elution solvent strength from the stationary phases that caused the peak broadening in ion-exchange mode. On PET-PEI/BUDGE fibers, crosslinking increased the surface density of PEI, providing better coverage and minimizing the interactions with the surface. Also, the hydrophilic BUDGE crosslinker created a barrier toward further surface interaction. Hence, the BUDGE treatment in the surface modifications improved the ion-exchange protein separations on the PEI-modified PET channeled fiber columns.

Although the peak shapes and the resolution from the PET-PEI/BUDGE column was better than from the PET-PEI, all of the peaks suffer from broadening and strong tailing. The chromatograms in FIG. 13A-13C indicate that there was no significant change of the peak shapes (on a volume basis) at increased linear velocities, which suggest that the peak broadening was probably neither due to the longitudinal diffusion (the B term in Van Deemter equation) nor the mass transfer resistance (the C term in Van Deemter equation). Actually, the peak broadening and asymmetry was mainly from the non-ideal packing.

Unlike the PEI-modified PET fibers, nylon 6 fibers have both anion- and cation-exchange sites on the surface, the proportion of which varies as a function of pH as depicted in the upper portion of FIG. 14. As a result, the elution order of the three proteins was different for nylon 6 (FIG. 13C) than from the PEI-modified PET. When the elution gradient was applied, hemoglobin eluted first (actually not retained), followed by BSA and lysozyme. The p/ of BSA, hemoglobin, and lysozyme are 4.70, 6.80, and 11.35, respectively. The difference between the p/ of each protein and the environmental pH reflects the magnitude of the net charge of the proteins. (Of course, p/ reflects a global charge and not necessarily what is accessible for electrostatic interactions with the stationary phase.) As shown in FIG. 14, the p/ of BSA is 2.60 units below the buffer pH (7.5), while the p/ of lysozyme is 3.85 units above the buffer pH. This difference makes lysozyme significantly more retentive than BSA, though nylon carries more cationic sites than anionic sites (0.29 units difference, according to FIG. 14). The p/ of hemoglobin is only 0.70 units below the buffer pH; thus, it is relatively charge-neutral in comparison to a surface that is effectively charge-neutral at this pH. While electrostatic interactions can explain the retention of the proteins, the interactions are surely more complicated, including factors such as hydrophobic interactions.

One point relative to the retention of the proteins on nylon 6 is reflective of the relatively low amine density of that surface (Table 2). As seen in FIG. 13C, the peak area of the BSA elution (second peak) is significantly lower than the other two peaks. The reduced elution peak area for BSA is due to the fact that some portions of the injected BSA co-elute with the unretained hemoglobin. The two distinct elution peaks of BSA on the nylon 6 column were also observed in the single-protein injection experiment. In this case, it seems clear that the lower density of amines on the nylon 6 surface, in combination with the low p/ of BSA, yielded a very low binding capacity. As a result, some portions of the injected BSA were totally unretained and eluted in the injection volume. In comparison, lysozyme, further removed from its p/ value, was readily retained, much like hemoglobin and BSA on the PEI phases.

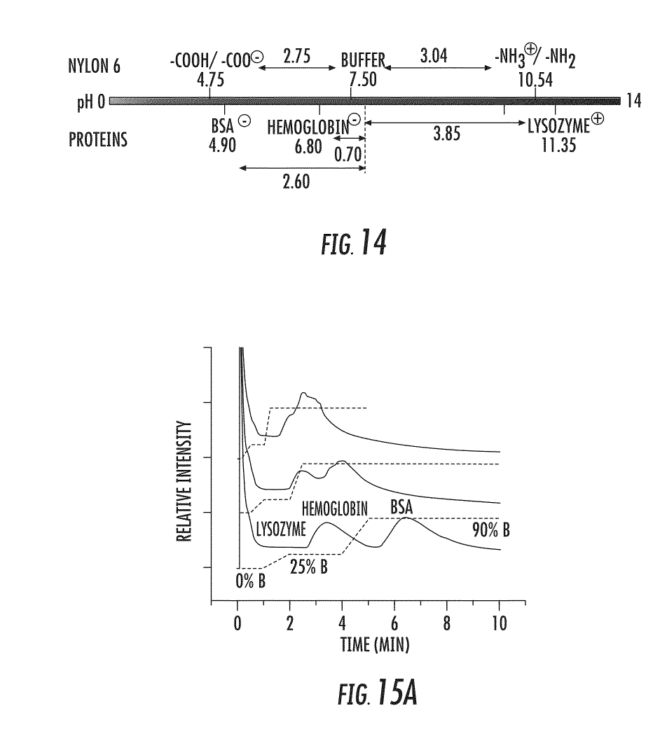

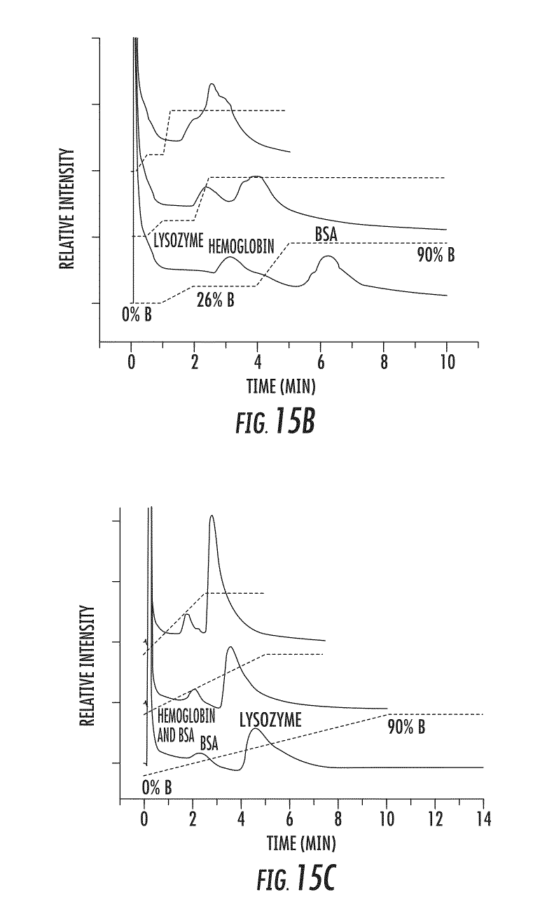

FIGS. 15A-15C show the elution profiles of the three proteins at the same flow rates but with different gradient rates. In each case, the slowest gradient (bottom trace) represents the optimum gradient program for the respective phase, with the rates increased by a factor of 4 from bottom to top separations. There were significant differences in terms of both the peak resolution and recoveries between the PEI-modified and native nylon 6 fiber columns. In comparing the PET-PEI and PET-PEI/BUDGE responses (FIG. 15A, FIG. 15B), no appreciable differences were seen between the two as higher gradient rates are applied. As expected, increases in the gradient rates (shorter times) led to substantial sacrifices in the observed resolution for the modified fibers, with the latter showing somewhat better resolution. In the case of the PET-PEI separations, the resolution decreased from 1.36 to ˜0.6 for the critical pair, while for the BUDGE-modified system, it decreased from 2.1 to ˜0.6. This general phenomenon stems from the fact that the proteins do not desorb at a finite mobile phase composition, but over some range as dictated by the multiplicity of potential protein-surface interactions. Therefore, as the rate is increased, the ranges “overlap” in time as reflected in chromatograms.

In the case of the nylon 6 column (FIG. 15C), the resolution did not visually appear to suffer as much as the gradient is increased. In fact, different from the modified phases, the resolution showed no real degradation (admittedly, the gradients used for nylon 6 are much slower than explored for the modified fiber phases, which are optimized for the modified fiber phases). More pronounced was a loss in protein recovery as reflected in the absorbance peak heights from the nylon column as the gradient time was increased. Quantitatively, the integrated peak areas decreased when the gradient time was increased. The losses of protein may be the result of protein unfolding on the stationary phase, the hydrophobic interactions between the proteins and the native nylon fiber surface may cause protein unfolding/denature, leading to potential irreversible binding as well as peak broadening, the extent of which may increase with the protein residence time on-column. Thus, any advantage expected from using slow gradients was lost in the case of nylon 6; in fact, the resolution across this 4× difference in gradient rate did not change, ranging from 1.35 to 1.4. On the PET-PEI and PET-PEI/BUDGE, the peak profiles do not reflect protein unfolding or irreversible binding as witnessed by the uniform recoveries. Here, the ionic interactions dominate potential nonionic (hydrophobic) interactions between proteins and stationary phase, resulting in favored desorption kinetics under ion-exchange chromatography conditions.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

What is claimed is:

1. An apparatus, comprising:
a fluid conduit having a first end and a second end disposed opposite said first end;
a polymeric support phase disposed within said conduit between said first end and said second end; and
a stationary phase at a surface of the support phase, the stationary phase comprising a crosslinked polymer network.

2. The apparatus of claim 1, wherein the polymeric support phase comprises one or more fibers.

3. The apparatus of claim 2, wherein the one or more fibers comprise a plurality of co-linear channels, each of which extending along the longitudinal length and along the exterior surface of the fiber.

4. The apparatus of claim 3, wherein each said channel of said fiber(s) extends helically around each fiber.

5. The apparatus of claim 1, wherein the polymeric support phase comprises poly(ethylene terephthalate), a polyamide, or polypropylene.

6. The apparatus of claim 1, the stationary phase comprising one or more of amine functionality, imine functionality, carboxylate functionality, hydroxyl functionality, thiol functionality, or combinations thereof.

7. The apparatus of claim 1, wherein the crosslinked polymer network comprises crosslinked polyethylene imine.

8. The apparatus of claim 1, wherein the crosslinked polymer network has been crosslinked with a crosslinking agent comprising epoxide functionality, isocyanates functionality, hydroxyl functionality, carbodiimide functionality, amine functionality, carboxylic acid functionality, acid halide functionality, or combinations thereof.

9. The apparatus of claim 1, wherein the crosslinked polymer network has been crosslinked via reaction with 1,4-butane diglycidyl ether as crosslinking agent.

10. The apparatus of claim 1, wherein the apparatus comprises multiple layers of the stationary phase at the surface of the support phase.

11. A polymeric support phase including a fiber defining a plurality of co-linear channels on a surface of the fiber, each channel extending along the longitudinal length of the fiber, each channel extending along the exterior length of the fiber, the fiber further comprising a crosslinked polymer network at a surface of the fiber.

12. The polymeric support phase of claim 11, wherein the fiber is formed of a polymeric composition that includes poly(ethylene terephthalate), a polyamide, or polypropylene.

13. The polymeric support phase of claim 11, the crosslinked polymer network comprising one or more of amine functionality, imine functionality, carboxylate functionality, hydroxyl functionality, thiol functionality, or combinations thereof.

14. The polymeric support phase of claim 11, wherein the crosslinked polymer network comprises crosslinked polyethylene imine.

15. The polymeric support phase of claim 11, wherein the crosslinked polymer network has been crosslinked with a crosslinking agent comprising epoxide functionality, isocyanates functionality, hydroxyl functionality, carbodiimide functionality, amine functionality, carboxylic acid functionality, acid halide functionality, or combinations thereof.

16. The polymeric support phase of claim 11, wherein the crosslinked polymer network has been crosslinked via reaction with 1,4-butane diglycidyl ether as crosslinking agent.

17. A method, comprising the steps of:
providing a fluid conduit having a first end and a second end disposed opposite said first end and with a polymeric support phase disposed within said conduit between said first end and said second end, said support phase comprising a stationary phase on a surface of the support phase, the stationary phase comprising a crosslinked polymer network;
moving fluid containing a species through said conduit; and
separating said species from said fluid by chemical attachment of said species to said stationary phase in said conduit.

18. The method of claim 17, further comprising using an instrument disposed at said second end of said conduit to detect said species.

19. The method of claim 17, further comprising removing the support phase from the conduit after a predetermined duration of movement of the fluid through the conduit.

20. The method of claim 17, further comprising removing the species from the stationary phase and collecting said species.
